(19) United States
(12) Patent Application Publication
(10) Pub. No.: US 2014/0303522 A1
(43) Pub. Date: Oct. 9, 2014

(54) SCOLIOSIS EVALUATION SYSTEM AND EVALUATION APPARATUS APPLIED TO THE SAME SYSTEM

(71) Applicant: TOYO UNIVERSITY, Bunkyo-ku, Tokyo (JP)

(72) Inventors: Toshinari Akimoto, Kawagoe-shi (JP); Nobuyuki Terada, Nirasaki-shi (JP)

(21) Appl. No.: 14/361,079

(22) PCT Filed: Nov. 29, 2012
(86) PCT No.: PCT/JP2012/080860

§ 371 Date: May 28, 2014

(30) Foreign Application Priority Data
Nov. 29, 2011 (JP) 2011-260530

(51) Int. Cl. A61B 5/00; A61B 6/00; A61B 5/107

(52) U.S. Cl. CPC A61B 5/4566; A61B 5/1077; A61B 5/742; A61B 6/505; A61B 5/1079; A61B 5/7282
USPC 600/594

(57) ABSTRACT

The scoliosis evaluation system comprises a 3D sensor (100) that takes the back of a subject and acquires the 3D data thereon; a characteristic part designator (102) that designates a characteristic part of which the degree of curvature is to be measured on the back of the subject, an uneven state detector (103) that detects an uneven state of a body surface in a horizontal direction based on the 3D data on the characteristic part designated by the characteristic part designator, and a display monitor (200) that displays a result detected by the uneven state detector.

### CROSS REFERENCE TO RELATED APPLICATION

This invention is based upon and claims the benefit of priority under 35 U.S.C. §119 to International Patent Application No. PCT/JP2012/080860, filed on Nov. 29, 2012, the entire content of which is incorporated herein by reference.

### TECHNICAL FIELD

The present invention relates to scoliosis evaluation systems and evaluation apparatuses applied to the same systems, and in particular, relates to a scoliosis evaluation system that quantitatively evaluates scoliosis in a simple way and at low cost and an evaluation apparatus applied to the same system.

### BACKGROUND ART

Spine scoliosis (hereinafter referred to as “scoliosis”) is a disease such that the spinal column (backbone) curves laterally and/or is twisted. This disease is particularly common in women.

Because the curvature of the spinal column is usually associated with the twist thereof, the progress of symptom results in the projection of ribs. Furthermore, severe curvature produces lumber backache, compression and deformity of rib cage, and has harmful effects on inner organs such as breathing problem, and circulatory disease.

The diagnosis of scoliosis needs a quantitative measurement. In particular, it is necessary to quantitatively measure to judge whether a surgery should be performed or evaluate the degree of symptomatic improvement due from treatment.

Conventional diagnoses of scoliosis have been performed through an X-ray inspection method with the use of an X-ray photographic apparatus. In the X-ray inspection method, doctors take a chest X-ray image to determine the degree of curvature of the spinal column. FIG. 11 shows an example of an image taken by an X-ray photographic apparatus. As shown in FIG. 11, the doctors can look at the degree of lateral curvature of the spinal column.

However, general X-ray inspection methods can determine the degree of lateral curvature, but the methods cannot determine the degree of unevenness on the body surface. Of course, the degree of unevenness can be determined by taking X-ray images from various directions such as a side of a subject. However, because there is a risk for X-ray exposure, it is desirable to avoid multiple X-ray taking as much as possible.

Alternatively, there is a measurement method using a CT (Computer Tomography) scanner. However, because the CT scanner is expensive and large-scaled, it is difficult to introduce it into small hospitals. Furthermore, because CT scan inspection is performed under that condition that a subject lays down, the state of the spinal column and the lib is changed by the effect of gravity. It is therefore difficult to accurately evaluate the symptoms of scoliosis. In addition, as is the case in the X-ray inspection method, the measurement method using a CT scanner has a risk of exposure.

Patent document 1 discloses a technology of a body distortion detection apparatus without the use of an X-ray photographic apparatus and a CT scanner.

This detection apparatus has measuring means composed of the first and second sensors. Each sensor is attached on the right or left upper arm of a subject to measure the 3D posture of the sensor. First, this apparatus determines the posture of the right and left arms from the data obtained after completion of the motion of the right and left arms. Next, the apparatus determines parts of the upper body where has strong muscles depending on the posture difference between the right and left arms.

### PRIOR ART DOCUMENTS

### Patent Documents

[Patent Document 1] Japanese Patent Document 2010-207399

### SUMMARY OF THE INVENTION

### Problems to be Solved

However, the body distortion detection apparatus according to patent document 1 cannot apply to a quantitative measurement of scoliosis.

Therefore, as an inspection method without the use of an X-ray photographic apparatus and a CT scanner, methods applying moiré method have been used for measuring the shape of body surface.

The moiré method is a method for three-dimensionally measuring the shape of body surface form by using an interference pattern of light.

For instance, as shown in FIG. 13, a moiré imaging apparatus requires a moiré image of the back of a subject (human body) H. The apparatus is configured to measure the bilateral difference “h” of peaks in characteristic parts H1 to H6 of which the degree of curvature should be measured, and evaluate the degree of curvature based on the ratio of bilateral difference “h” to shoulder width “d”.

This method has the advantage that a subject is not exposed to radiation by inspection in contrast to measurement methods with the use of an X-ray photographic apparatus and a CT scanner are used. In addition, the method is a non-invasively measurement method.

However, apparatuses with the use of moiré method require a lot of equipment and have a large size, and therefore they are very expensive (for instance, there is an apparatus that requires a cost over ¥1,000,000).

In addition, because it is technically difficult to perform moiré images processing in computers, the evaluation of scoliosis based on moiré images is manually done by doctors and/or engineers, and therefore this evaluation suffers from low efficiency and restriction on the number of subjects per unit time.

Furthermore, because the evaluation method based on moiré images includes a measurement process by hand as described above, this evaluation method has a large margin of measurement error depending on the skills of engineers and had a low accuracy.

There are a large number of scoliosis patients. For instance, it is estimated that 20,000 to 40,000 children with sudden scoliosis exist in Japan. Therefore, the improvement of inspection efficiency and accuracy is needed in Japan and elsewhere.

In addition, there is a need for development of a scoliosis evaluation system in a simple way and at low cost to easily conduct an inspection of scoliosis as in physical examination at school in the miscellaneous category.

To respond to such a request, the present invention intends to provide a scoliosis evaluation system that quantitatively evaluates scoliosis in a simple way and at low cost and an evaluation apparatus applied to the same system.

### Solution to the Problems

To address the issue, there is provided a scoliosis evaluation system comprising: a 3D data acquirer that takes the back of a subject to acquire the 3D data thereon; a characteristic part designator that designates a characteristic part of which the degree of curvature is to be measured on the back of the subject; an uneven state detector that detects an uneven state of a body surface in a horizontal direction based on the 3D data on the characteristic part designated by the characteristic part designator; and a display that displays a result detected by the uneven state detector.

In addition, there is provided an evaluation apparatus applied to the scoliosis evaluation system according described above, comprising: a horizontal plate that is placed in contact with a surface of the lumber part of the subject in a horizontal direction; and a pair of lateral plates that is extended in a direction perpendicular to the horizontal plate and is placed in contact with bilateral parts of the lumber part of the subject.

### Effect of the Invention

This invention can provide a scoliosis evaluation system that quantitatively evaluates scoliosis in a simple way and at low cost and an evaluation apparatus applied to the same system.

### BRIEF DESCRIPTION OF THE DRAWINGS

FIG. 1 is a block diagram showing a construction of a scoliosis evaluation system according to an embodiment of the present invention.

FIG. 2 is a figure showing an example of apparatus configuration of the scoliosis evaluation system shown in FIG. 1.

FIG. 3 is an outline view showing a 3D sensor applied to the scoliosis evaluation system shown in FIG. 1.

FIG. 4 is a view explaining the measurement principle of the 3D sensor shown in FIG. 3.

FIG. 5 is a view explaining the measurement principle of the 3D sensor shown in FIG. 3.

FIG. 6 is a view explaining the measurement principle of the 3D sensor shown in FIG. 3.

FIG. 7 is a flowchart showing the procedure of evaluating scoliosis implemented in the scoliosis evaluation system shown in FIG. 1.

FIG. 8 is a view showing a display example of a result measured by the scoliosis evaluation system shown in FIG. 1.

FIG. 9 is a view explaining a measurement result of a health subject and a scoliosis patient by the scoliosis evaluation system shown in FIG. 1.

FIG. 10 is a view showing a case of scoliosis.

FIG. 11 is an example of an image taken by an X-ray photographic apparatus.

FIG. 12 is a view explaining the bilateral difference in the state that the spinal column is twisted by scoliosis.

FIG. 13 is a view showing an evaluation example of scoliosis using moiré method.

FIG. 14(A) is a back elevational view showing the skeleton framework of an evaluation apparatus. FIG. 14(b) is a view showing a skeleton framework of an evaluation apparatus according to an embodiment of the present invention.

### EMBODIMENTS OF THE INVENTION

An embodiment of the present invention will be described below with reference to accompanying drawings in detail. Here, identical members in the drawings are indicated with the same reference numerals respectively, and redundant descriptions are eliminated.

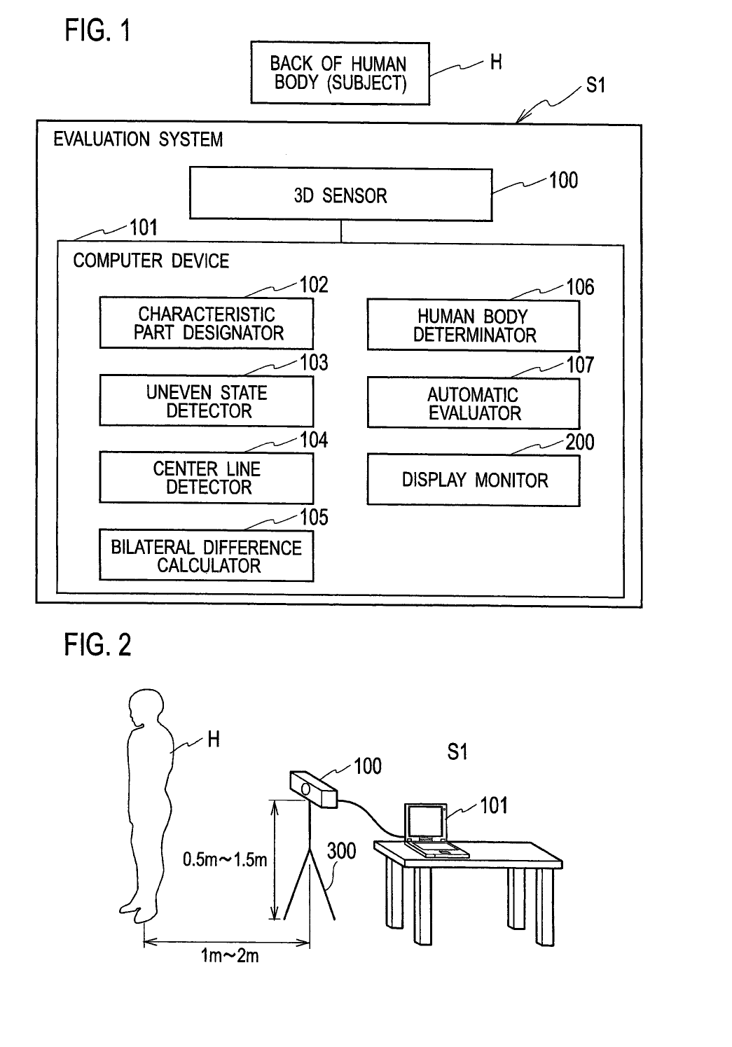

As shown in the block diagram of FIG. 1, a scoliosis evaluation system S1 related to this embodiment includes a 3D sensor (or 3D camera) 100 (3D data acquiring means) that takes the back of a subject H to acquire its 3D data, a characteristic part designator 102 (characteristic part designating means) that designates a characteristic part of which the degree of curvature is to be measured on the back of the subject H, an uneven state detector 103 (uneven state detecting means) that detects an uneven state of a body surface in a horizontal direction based on the 3D data on the characteristic part designated by the characteristic part designator 102 and a display monitor 200 (displaying means) that displays a result detected by the uneven state detector 103.

The display monitor 200 is also adapted so as to display peak positions of unevenness acquired based on the result detected by the uneven state detector 103.

The scoliosis evaluation system S1 further includes a center line detector 104 (center line detecting means) that detects a center line of the back of the subject H along a vertical direction based on the 3D data acquired by the 3D sensor 100 and a bilateral difference calculator 105 (bilateral difference calculating means) that calculates the bilateral difference between peak positions on the left and right sides of the center line in the characteristic part designated by the characteristic part designator 102.

The display monitor 200 is also adapted so as to display the result calculated by the bilateral difference calculator 105 in addition to the result detected by the uneven state detector 103.

Furthermore, the scoliosis evaluation system S1 further includes a human body determinator 106 (human body determining means) that determines whether the 3D data acquired by the 3D sensor 100 relates to a human body or not.

In addition, the scoliosis evaluation system S1 includes an automatic evaluator 107 (evaluating means) that evaluates the degree of scoliosis in the subject H by comparison with a threshold preliminarily set for at least one of the result detected by the uneven state detector 103 and the result calculated by the bilateral difference calculator 105.

Then, the display monitor 200 is adapted so as to display the result evaluated by the evaluator 107 in addition to the result detected by the uneven state detector 103.

Note that, in this embodiment, a program (software) executed by a computer device 100 comprised of a personal computer or the like constitutes the characteristic part designator 102, the center line detector 104, the bilateral difference calculator 105, the human body determinator 106 and the automatic evaluator 107.

Further, the 3D sensor 100 and the computer device 100 are connected to each other through a USB cable or the like.

A TOF (time-of-flight) 3D sensor can be applied for the 3D sensor 100.

The TOF type 3D sensor irradiates near infrared light (LED light) actively and measures a distance with the use of its reflected light.

In detail, the above sensor modulates the pulse of invisible light, such as infrared light, then irradiates the pulse-modulated light within its field angle and finds out a reciprocating distance by measuring a phase delay of this pulse on the side of the image sensor.

Conventionally, this TOF type 3D sensor was an expensive instrument costing about ¥5,000,000 to about ¥10,000,000. But, with the progress of inexpensiveness in recent years, there is developed a camera less than several tens of thousands yen.

In addition, a 3D sensor employing a laser-pattern projection method can be applied for the 3D sensor 100.

In a constitutional example shown in FIG. 2, there is used the 3D sensor 100 employing this laser-pattern projection method.

The 3D sensor 100 adopting the laser-pattern projection method irradiates an infrared pattern to a target object and acquires a range image by means of triangular surveying.

More concretely, a “Kinect (Microsoft trademark)” sensor made by Microsoft Co. Ltd. can be applied for the 3D sensor adopting the laser-pattern projection method. At first, this “Kinect” sensor was provided for a game console, but it may be also connected to the computer device (personal computer) 101 through a USB terminal.

Then, when using a software named “Kinect for Windows (trademark) SDK (Software Development Kit)” provided by Microsoft Research Co. Ltd., the Kinect sensor can be controlled by the computer device 101 through a program written in C language.

This “Kinect” sensor is available for the price of about ten-odd thousands yen (about one hundred and several tens of dollars), allowing the cost of this evaluation system S1 to be reduced.

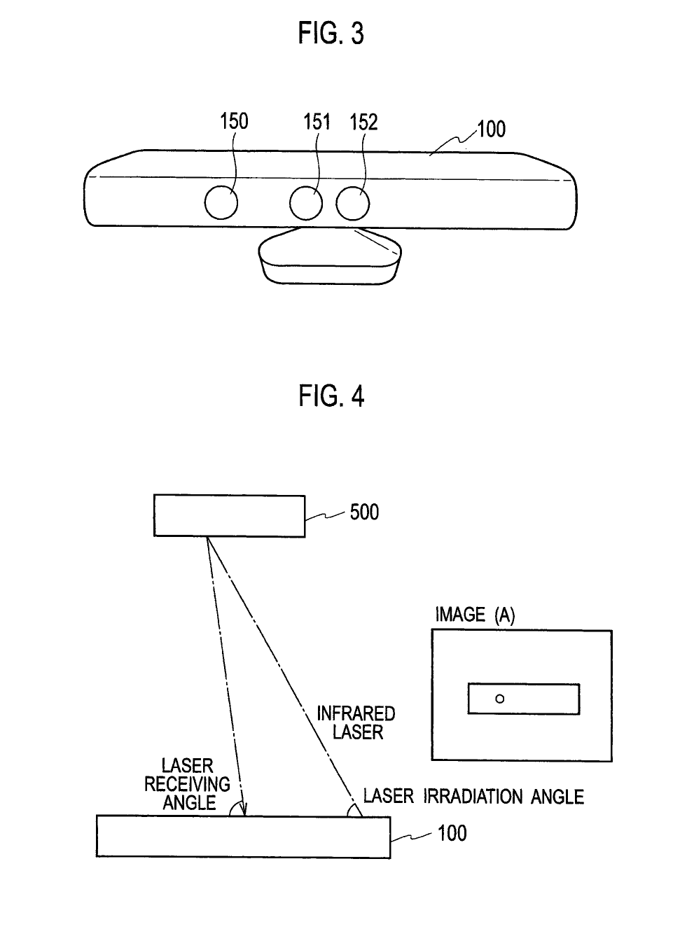

FIG. 3 illustrate an outline view of the 3D sensor (“Kinect” sensor) 100. The 3D sensor 100 is equipped with an infrared-laser emitting part 150, a RGB color image recognition camera 151 and an infrared camera 152 for measurement of depth.

Note that the 3D sensor 100 includes an electric tilt mechanism allowing the sensor to swivel by 30 degrees in the vertical direction. It can be therefore adjusted to the height of the subject H etc. by the side of the computer device 101.

In the 3D sensor 100, there are built-in a triaxial acceleration sensor, a DDR2/SDRAM main memory of 64 MB, a signal conditioning processor and so on.

For more accurate measurement, the 3D sensor 100 may be provided with a water level for adjustment of levelness etc.

As shown in FIG. 2, this evaluation system S1 can be comprised of the 3D sensor 100 attached to a tripod 300 capable of height adjustment (height controllable in the range of e.g. 0.5 to 1.5 m) and a note-type personal computer 101 installing a program (software) capable of realizing respective functions of the characteristics part designator 102, the uneven state detector 103, the center line detector 104, the bilateral difference calculator 105, the human body determinator 106, the automatic evaluator 107 etc.

Note that the distance between the 3D sensor 100 and the subject H is advantageously set from about 1 m to 2 m.

Here, the measurement principle of the 3D sensor 100 adopting the laser-pattern projection method will be described with reference to FIGS. 4 to 6, in brief.

As shown in FIG. 4, an infrared laser irradiated from the infrared-laser emitting part 150 (see FIG. 3) of the 3D sensor 100 at a constant irradiation angle is reflected on an object 500 and subsequently enters the “depth-measurement” infrared camera 152 (see FIG. 3) for detecting the laser. In this case, the distance from the sensor to the object 500 can be calculated by a base and angles at both ends of the base. Note that an image of the object 500 onto which the infrared laser has been irradiated is illustrated as an image (A). Further, a laser receiving angle is measured by the image.

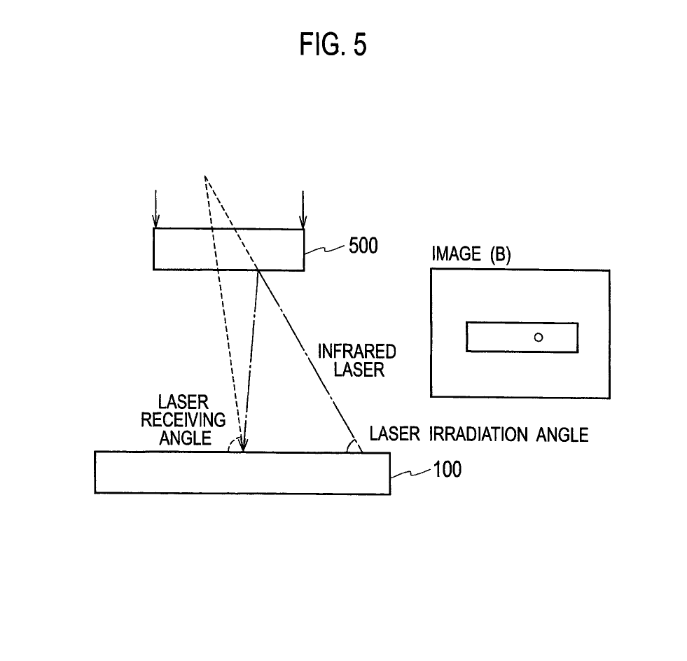

Also, as shown in FIG. 5, even when the object 500 moves toward the 3D sensor 100, the distance from the sensor to the object 500 can be calculated by a base and angles at both ends of the base similarly. Note that an image of the object 500 onto which the infrared laser has been irradiated is illustrated as an image (B). Further, a laser receiving angle is measured by the image.

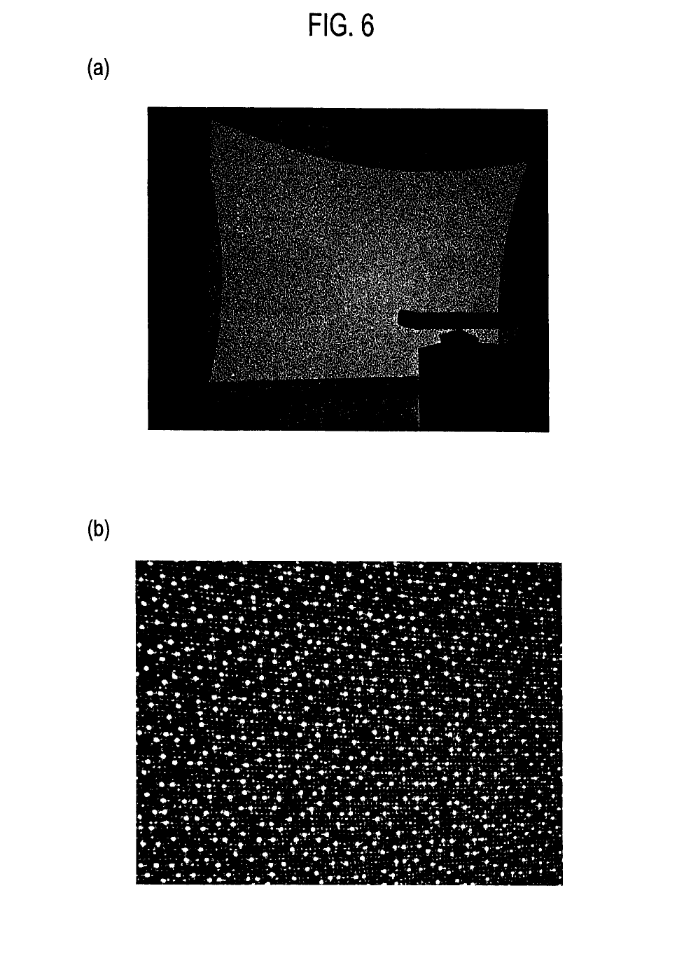

As shown in FIGS. 6(a) and 6(b), the above-mentioned Kinect sensor irradiates a known optical pattern within the angle of view in advance and subsequently restores a 3D structure of the object by the degree of geometric distortion of the pattern. For instance, a method comprising the steps of once diffusing a light source through a diffuser plate and subsequently creating a projection pattern with the use of a transparent plate lining up micro-lenses, would be applied to the restoration.

Note that the 3D sensor 100, such as Kinect sensor, is capable of acquiring motion pictures besides still images. Although the evaluation system S1 related to this embodiment is essentially directed to evaluate scoliosis on the basis of still images, it is also possible to evaluate scoliosis comprehensively by allowing the subject to perform a given action and successively acquiring the subject's situation in the form of motion pictures.

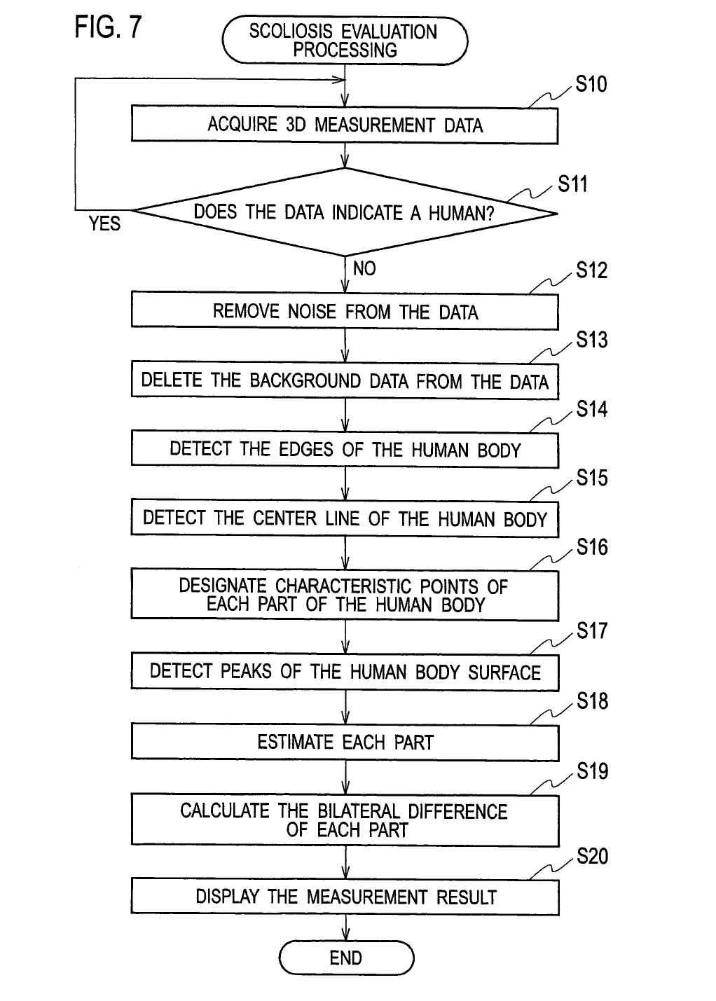

Next, the scoliosis evaluation processing procedures executed by the evaluation system S1 related to the embodiment will be described with reference to a flow chart of FIG. 7.

At step S10, 3D measurement data is acquired by the 3D sensor 100 that takes a picture of the back of a subject H. In reality, the acquired 3D measurement data is stored in a built-in hard disc drive, memories, etc. in the computer device 101.

At step S11, the human body determinator 106 judges whether the related 3D measurement data indicates a human body or not, based on the acquired 3D measurement data.

Then, if the judgment is “No”, the routine returns to step S10, and if the judgment is “Yes”, the routine goes to step S12.

Concretely, the judgment of human body at step S11 can be realized by previously storing a pattern of the back of a human body and subsequently executing the pattern matching between the pattern and the 3D measurement data. By executing such a human body judgment, it is possible to automate subsequent processes. That is, under condition of continuing the acquisition process of the 3D measurement data through the 3D sensor 100, if the subject H takes a predetermined posture while directing the back toward the 3D sensor 100, then it is judged that the related 3D measurement data belongs to the human body. With this judgment, the routine can be shifted to subsequent steps, allowing an execution of effective inspection.

Next, at step S12, it is executed to remove noise from the acquired 3D measurement data with the use of filters.

At step S13, it is executed to delete the background data from the 3D measurement data.

Based on the 3D measurement data, at step S14, the edges of the image are detected to acquire an outline of the back of the subject H.

At step S15, the center line detector 104 detects a center line of the back of the subject H (human body). As this center line acts as a reference for evaluating the back of the subject H while separating it into left and right, it is important to determine how to detect the center line.

The following method will be expected for how to detect the center line.

It is possible to detect a vertical center line from the width of the back based on the outline of the back of the subject H acquired at step S14. More specifically, it is expected to acquire, based on the outline, widths at respective positions of the back of the subject H and subsequently establish a center line by connecting respective mid-points (½ positions) of these widths to each other.

Alternatively, the vertical center line may be detected on the basis of an uneven state of the back of the subject H detected by the uneven state detector 103. In detail, since the spinal column is positioned under a concave state in the back of a human body, it is suspected to provide a center line by joining designated positions, which constitute respective concave bottoms positioned roughly in the center of data representing the uneven state of the back of the human body, to each other.

Alternatively, the vertical center line may be detected by superimposing X-ray photography image data of the back of the subject H, which has been taken separately, on the 3D measurement data. That is, when the 3D measurement data alone are not sufficient for grasping a center line or when higher-accuracy evaluation is desired, a vertical center line could be determined by referring to X-ray photography image.

Alternatively, the vertical center line may be detected by superimposing moiré image data of the back of the subject H, which has been taken separately, on the 3D measurement data. That is, when the 3D measurement data alone are not sufficient for grasping a center line or when higher-accuracy evaluation is desired, a vertical center line could be determined by referring to a moiré image.

Moreover, for instance, the center line may be determined by previously applying a marker, such as reflection tape, to the spinal column of the subject H and subsequently detecting the position of the marker with the use of an image taken by the RGB camera 151 (see FIG. 3) built in the 3D sensor 100.

Next, at step S16, the characteristic part designator 102 designates characteristic parts of which the degrees of curvature are to be measured on the back of the subject H

Regarding the destination of characteristic parts, there are expected one case where they are designated automatically and another case where they are manually designated by an operator.

On the assumption that data related to a characteristic part to be measured is registered in advance, for example, the degree of curvature about the same characteristic part may be measured for a plurality of examinees, based on the registered data. More specifically, on the assumption of regarding e.g. “seventh cervical vertebra” as the characteristic part, the position of “seventh cervical vertebra” of the subject H may be automatically detected on the basis of 3D measurement data or an image taken by the RGB camera 151 and subsequently, it may be carried out to measure the degree of curvature at that position. Similarly, “shoulder”, “lumber part” or the like may be registered as the characteristic part to perform a measurement automatically.

Alternatively, an operator of the scoliosis evaluation system S1 or a doctor may be in charge of designating a body part, of which the degree of curvature is to be measured, as the characteristic part by manipulating a pointing device, such as mouse and track pad. Alternatively, selection buttons of “seventh cervical vertebra”, “shoulder”, “lumber part”, etc. may be previously displayed on a display panel so that the characteristic part can be designated by an operator or a doctor who selects one of the selection buttons with the use of such a pointing device.

Next, at step S17, the uneven state detector 103 detects the uneven state of a body surface in the horizontal direction on the basis of the 3D measurement data about the characteristic parts designated at step S16 and detects a peak position of the body surface on the basis of the uneven state.

Consequently, it is possible to grasp how the spinal column, ribs, or the like is twisted by scoliosis, which could not be grasped by the conventional X-ray inspection, with ease.

Next, at step S18, it is executed to estimate each part of the back of the subject H, based on the 3D measurement data. Consequently, upon an estimation of the position of e.g. a lumber part or breech, its part can be established as a reference for twist. That is, as the lumber part or breech can be generally regarded as a part forming a substantially-horizontal plane, it is possible to establish this lumber part or breech as a reference for the degrees of twist of the characteristic parts, such as “seventh cervical” and “shoulder”.

At step S19, the bilateral difference calculator 105 calculates a difference in height between left and right peak positions interposing the above center line therebetween, with respect to each characteristic part designated at step S16. As a result, it is possible to grasp the degree of twist of the characteristic parts.

At step S20, the measurement result is displayed on the display monitor 200 and the process is ended.

The display format is not limited to a specific one, and therefore any existing display format is applicable.

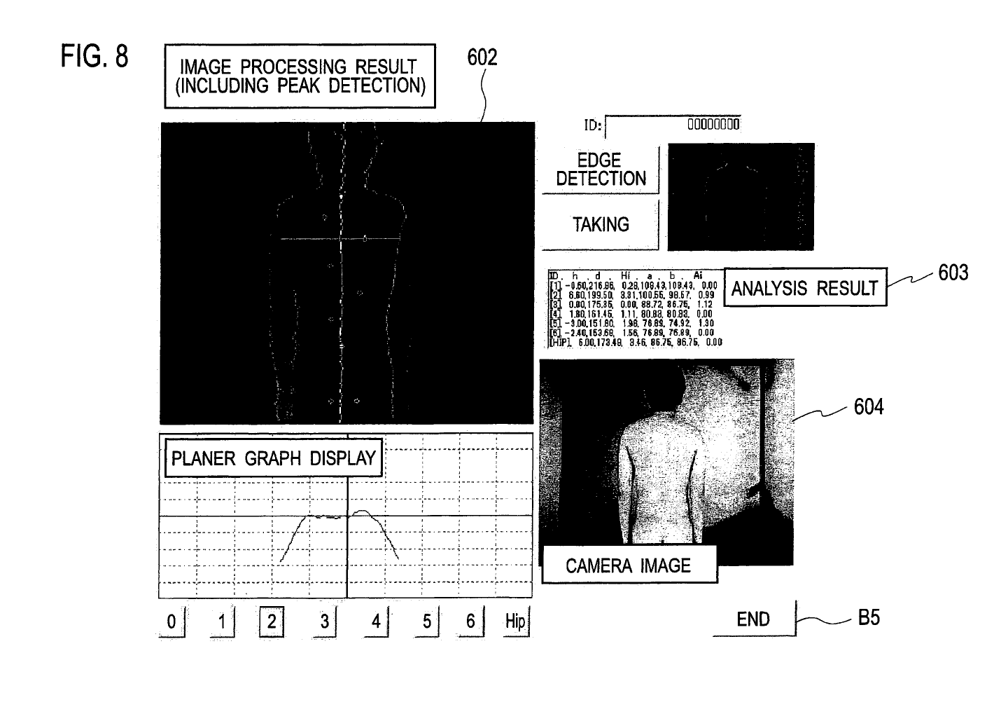

In the example shown in FIG. 8, there are displayed a planer graph display section 601, an image processing result 602 showing peak detection etc., an analysis result 603 consisting of an image and numerical data and a camera image 604 of the back of the subject H. In addition, although not shown, the display monitor may include a 3D data display section to display the 3D data in the form of a polygon or wire frame. Alternatively, the view point etc. of a 3D image may be changed by manipulating either a button or a pointing device.

In addition, the display monitor may be provided with a function of printing a displayed image or data.

Based on the measurement result on display, an operator of the scoliosis evaluation system S1 or a doctor can estimate whether or not the subject H is in the disease situation of scoliosis or how the scoliosis becomes advanced in the subject.

Owing to the automatic evaluator 107, it is also possible to automatically estimate whether or not the subject H is in the disease situation of scoliosis or how the scoliosis becomes advanced in the subject.

That is, by comparing at least one of the detection result by the uneven state detector 103 and the calculation result by the bilateral difference calculator 105 with a previously-set threshold value, it is possible to evaluate the degree of scoliosis of the subject H.

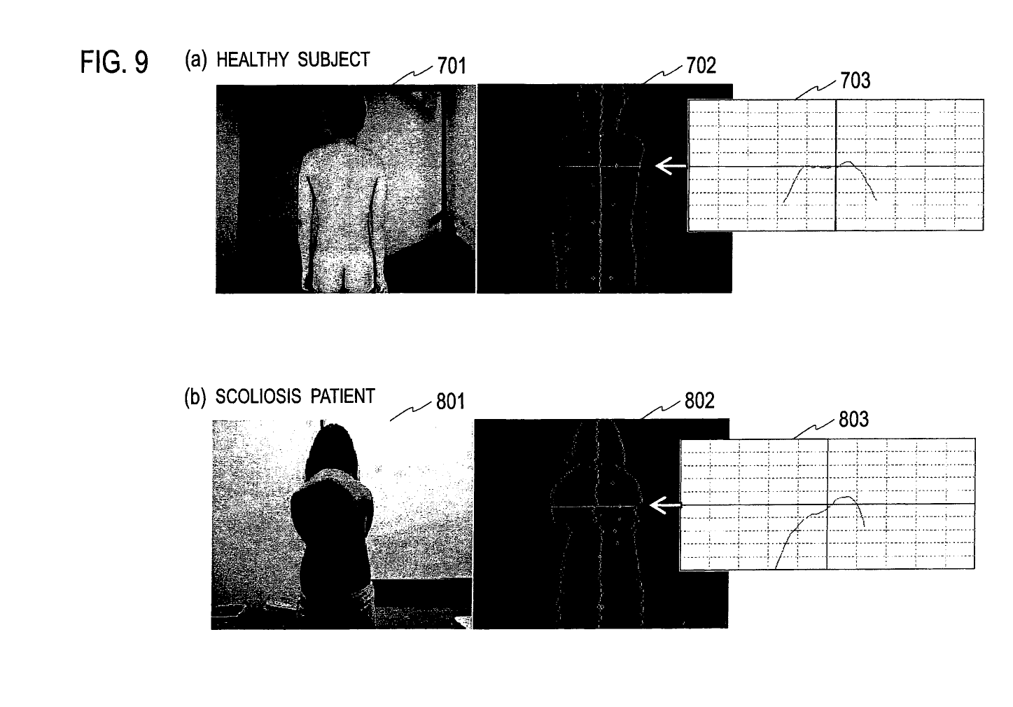

FIG. 9 shows the examples of measurement results. FIG. 9(a) shows the measurement result against a healthy subject, while FIG. 9(b) shows the measurement result against a scoliosis patient.

In FIG. 9(a), an image 701 of the back of an examinee (healthy subject), an image processing result 702 showing a peak of detection etc. and a plane graph 703 are displayed from the left.

In FIG. 9(b), an image 801 of the back of an examinee (scoliosis patient), an image processing result 802 showing a peak of detection etc. and a plane graph 803 are displayed from the left.

Although not shown, the display monitor may include a 3D data display section to display the 3D data in the form of a polygon or wire frame.

Based on the measurement results displayed as FIGS. 9(a) and 9(b), an operator of the scoliosis evaluation system S1 or a doctor then evaluates how the scoliosis is advancing in the subject, in a comprehensive manner. While, in case of executing an automatic evaluation, some messages, such as “Manifestation of Mild Scoliosis”, “Manifestation of Moderate Scoliosis (Estimated Necessity of Observation)” and “Manifestation of Severe Scoliosis (Estimated Necessity of Surgical Treatment)”, may be displayed if, for instance, the gradient of a graph in the plane graph 803 exceeds a preset threshold value.

Needless to say, the final determination, such as necessity/unnecessity of a surgery and necessity/unnecessity of various treatments for scoliosis, is performed by a medical specialist.

In connection, it is noted that data about the measurement results for the subject H contains personal information. Therefore, it is desirable to place strict control on the data, for example, with an establishment of passwords or the like when viewing the data.

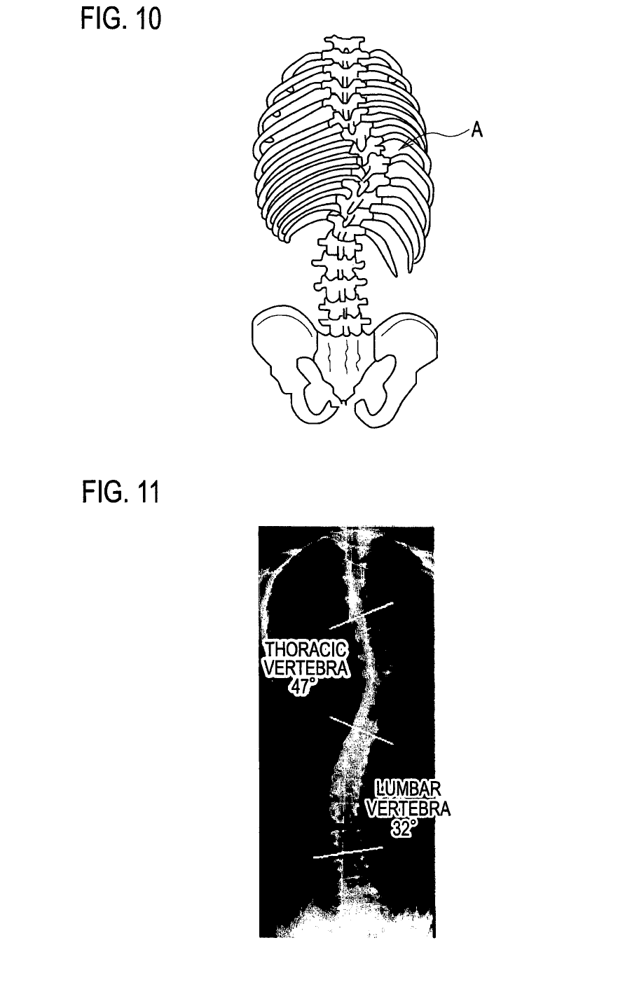

FIGS. 10 to 12 are reference materials showing a case of scoliosis.

In FIG. 10, referential mark “A” designates a curved point in the spinal column.

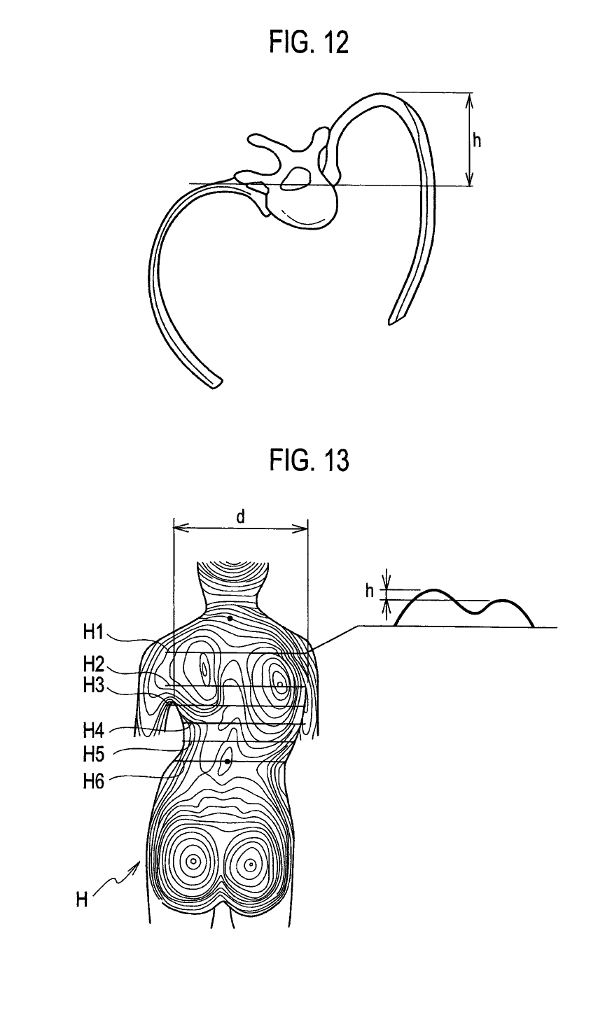

Further, the case shown in FIG. 12 has a given vertical difference h in plan view since a twist is caused in the spinal column and the ribs.

For such a case, the conventional X-ray inspection method cannot detect an uneven state of a body surface despite the capability of detecting the degree of curvature of the spinal column.

To the contrary, according to the scoliosis evaluation system S1 related to this embodiment, it is possible to grasp not only the degree of curvature of the spinal column about the case shown in FIG. 12 as well as an uneven state of a body surface easily and accurately, allowing the scoliosis to be evaluated appropriately.

As mentioned above, according to the scoliosis evaluation system S1 related to this embodiment, the following effects can be provided.

(1) Capability of Measuring Scoliosis in a Simple Way

Since this evaluation system S1 has reduced size and weight in comparison with the conventional apparatus, it becomes possible to go round with it and make measurements at various locations. In addition, as the same system does not require a particularly broad space, it allows the measurement in a consultation room etc.

(2) Capability of Quantitatively Measuring a Body-Surface Profile

Although the bending of spinal column measured by X-ray inspection has been estimated for the quantitative estimation of scoliosis, the estimation of a body surface profile has not been carried out quantitatively. If the body surface profile can be estimated quantitatively, then it becomes possible to perform not only a judgment in the necessity of a surgery and an estimation of an improvement after surgery but also early detection and treatment of disease situation.

(3) Capability of Measuring a Symptom of Scoliosis in a Natural State

That is, the position in actual question can be fairly evaluated because the measurement is not carried out under condition that the subject is horizontally postured like a measurement utilizing CT scanners, but instead the evaluation under condition that the subject is standing naturally.

(4) Capability of Confirming a Measuring Result on Site

As the measurement result of this evaluation system S1 is displayed in the measuring field, the measurement result can be utilized as materials for its confirmation with a patient in the measuring field, explanation about disease situation etc., so that the convenience of the system is enhanced.

(5) Capability of Providing a Scoliosis Evaluation System at a Low Price

According to this evaluation system S1, it can be expected to be widely used since the scoliosis evaluation system could be provided less costly than the conventional apparatus. Particularly, on the application to not only hospital etc. but also health checkup etc. conducted in schools in the miscellaneous category, the early detection and treatment of scoliosis in young adult can be accomplished.

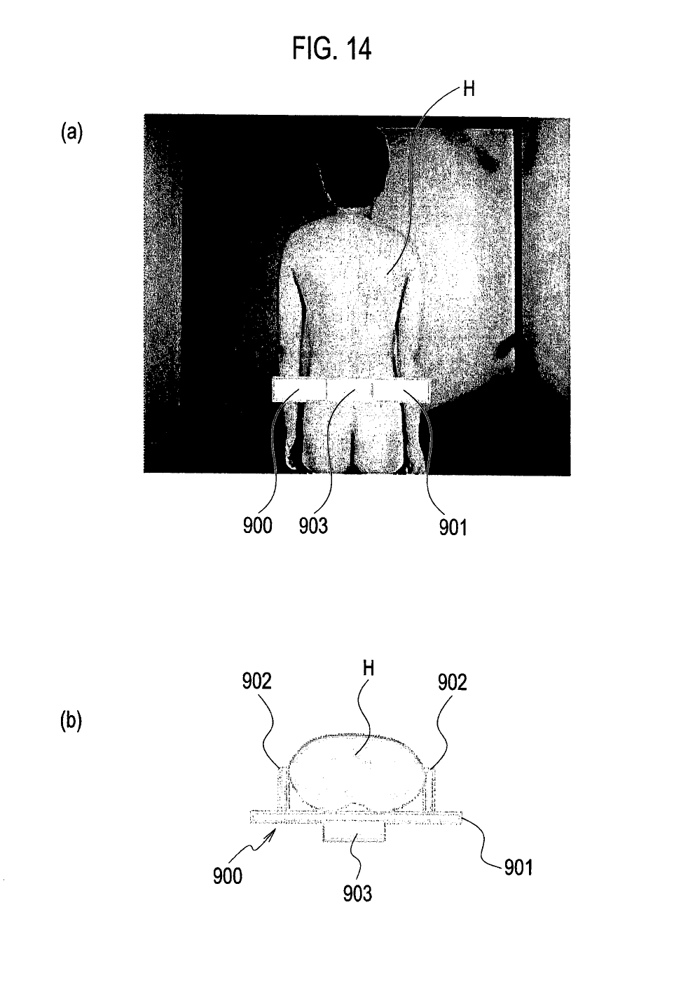

Next, an evaluation apparatus 900 applied to the above-mentioned scoliosis evaluation system S1 will be described with reference to FIG. 14.

The evaluation apparatus 900 includes a horizontal plate 901 that is placed in contact with a surface of the lumber part of the subject H in a horizontal direction and a pair of lateral plates 902 that are extended in a direction perpendicular to the horizontal plate and are placed in contact with both bilateral parts of the lumber part of the subject H.

Further, the horizontal plate 901 has a surface opposed to the 3D sensor and provided with a protrusion 903 with a given height (e.g. about 1 mm).

Note that the lateral plates 902 may be constructed so as to be movable in the horizontal direction, in conformity with the width of the lumber part of the subject H.

In case of using the so-constructed evaluation apparatus 900, it is possible to measure a reference for the degrees of twist of the above-mentioned characteristic parts, such as “seventh cervical” and “shoulder” precisely. That is, if the evaluation apparatus 900 is previously attached to a lumber part of the subject H to acquire the 3D measurement data through the 3D sensor 100, it is possible to acquire the data of the horizontal plane whose accuracy would be hard to obtain from a human body. Then, by adopting the 3D measurement data of the horizontal plane acquired by the evaluation apparatus 900 as a reference, it is possible to measure the degrees of twist of the characteristic parts more accurately.

In addition, by measuring the projection 903 on the horizontal plate 901 through the 3D sensor 100, it is possible to accomplish the calibration of 3D measurement.

Namely, with the provision of the projection 903, the 3D profile of the projection 903 is measured and successively, its measurement result is compared with actual data. Consequently, if there are nonconformities in terms of width, height and length therebetween, then a correction coefficient is calculated and subsequently, the measurement result is corrected by the calculated correction coefficient. Also, from a square profile that the projection 903 does have, the distortion of a lens of the 3D sensor 100 may be obtained and also used to correct the measurement result.

Although the present invention has been described with respect to one embodiment, the present invention is not limited to only this embodiment. Namely, the technical scope of the present invention should be interpreted in accordance with the appended claims absolutely and therefore, the present invention involves all modifications which are made by technique equivalent to the technique defined in the appended claims and which are included within the appended claims.

For example, a corrective tool for scoliosis patient etc. or a cushion for wheel chair may be designed on the basis of 3D measurement data acquired by the 3D sensor 100.

In addition, the body-mass index of scoliosis patient etc. may be evaluated on the basis of 3D measurement data acquired by the 3D sensor 100 and weight data by a weighing machine.

As for displaying of the measurement result, it may be further executed to emphasize the unevenness of a body surface based on the 3D measurement data so that an operator or a doctor can grasp the uneven state more easily.

In addition, it may be also executed to apply appropriate coloring to the unevenness of a body surface based on the 3D measurement data so that an operator or a doctor can grasp the uneven state more easily.

### INDUSTRIAL APPLICABILITY

The scoliosis evaluation system and the evaluation apparatus applied to the same system according to the present invention is able to appropriately evaluate scoliosis.

1. A scoliosis evaluation system comprising:
a 3D data acquirer that takes the back of a subject to acquire the 3D data thereon;
a characteristic part designator that designates a characteristic part of which the degree of curvature is to be measured on the back of the subject;
an uneven state detector that detects an uneven state of a body surface in a horizontal direction based on the 3D data on the characteristic part designated by the characteristic part designator; and
a display that displays a result detected by the uneven state detector.

2. The scoliosis evaluation system according to claim 1, wherein
the display displays peak positions of the uneven state acquired based on the result detected by the uneven state detector in addition to the result detected by the uneven state detector.

3. The scoliosis evaluation system according to claim 1, further comprising:
a center line detector that detects a center line of the back of the subject along a vertical direction based on the 3D data acquired by the 3D data acquirer;
a bilateral difference calculator that calculates the bilateral difference between peak positions on the left and right sides of the center line in the characteristic part designated by the characteristic part designator, wherein
the display displays the result calculated by the bilateral difference calculator in addition to the result detected by the uneven state detector.

4. The scoliosis evaluation system according to claim 1, wherein
the 3D data acquirer includes a 3D time-of-flight sensor.

5. The scoliosis evaluation system according to claim 1, wherein
the 3D data acquirer includes a 3D laser-pattern-projection sensor.

6. The scoliosis evaluation system according to claim 1, further comprising:
a human body determinator that determines whether the 3D data acquired by the 3D acquirer relates to a human body.

7. The scoliosis evaluation system according to claim 3, wherein
the center line detector detects the center line along the vertical direction from the width of the back based an outline of the back of the subject, wherein the outline is obtained from the 3D data.

8. The scoliosis evaluation system according to claim 3, wherein
the center line detector detects the center line along the vertical direction based on the uneven state of the back of the subject detected by the uneven state detector.

9. The scoliosis evaluation system according to claim 3, wherein
the center line detector detects the center line along the vertical direction by overlapping between the 3D data and X-ray photographic image data of the back of the subject, wherein the X-ray photographic data is separately taken.

10. The scoliosis evaluation system according to claim 3, wherein
the center line detector detects the center line along the vertical direction by overlapping between the 3D data and moiré image data of the back of the subject, wherein the moiré photographic data is separately taken.

11. The scoliosis evaluation system according to claim 3, further comprising:
an evaluator that evaluates the degree of scoliosis in the subject by comparison with a threshold preliminarily set for at least one of the result detected by the uneven state detector and the result calculated by the bilateral difference calculator.

12. The scoliosis evaluation system according to claim 11, wherein
the display displays the result evaluated by the evaluator in addition to the result detected by the uneven state detector.

13. An evaluation apparatus applied to the scoliosis evaluation system according to claim 1, comprising:
a horizontal plate that is placed in contact with a surface of the lumber part of the subject in a horizontal direction; and
a pair of lateral plates that is extended in a direction perpendicular to the horizontal plate and is placed in contact with bilateral parts of the lumber part of the subject.

14. The evaluation apparatus according to claim 13, wherein
the horizontal plate has a surface opposed to the 3D acquirer and provided with a protrusion with a given height for acquiring the 3D data.
